(19) United States
(12) Patent Application Publication
(10) Pub. No.: US 2024/0110885 A1
(43) Pub. Date: Apr. 4, 2024

(54) ELECTROCHEMICAL ANALYSIS OF REDOX-ACTIVE MOLECULES

(71) Applicant: B.G. NEGEV TECHNOLOGIES AND APPLICATIONS LTD., AT BEN-GURION UNIVERSITY, Beer Sheva (IL)

(72) Inventor: Hadar Shmuel BEN-YOAV, Ramat-Gan (IL)

(73) Assignee: B.G. NEGEV TECHNOLOGIES AND APPLICATIONS LTD., AT BEN-GURION UNIVERSITY, Beer Sheva (IL)

(21) Appl. No.: 18/262,518

(22) PCT Filed: Nov. 25, 2021
(86) PCT No.: PCT/IL2021/051401

§ 371 Date: Jul. 21, 2023

Related U.S. Application Data
(60) Provisional application No. 63/140,859, filed on Jan. 24, 2021.

(51) Int. Cl. G01N 27/27; G01N 27/30

(52) U.S. Cl. CPC G01N 27/27; G01N 27/30

(57) ABSTRACT

An electrochemical method of determining the presence and optionally the concentration of at least two analytes in a test sample which contains a mixture of analytes, wherein the analytes differ from one another in electrical charge and/or size, comprising the steps of: applying variable voltage, fixed voltage, current or impedance across an array of working electrodes consisting of electrodes coated with films of varying thickness and optionally a bare electrode, when the array is in contact with the test sample; measuring the current flowing or the impedance between each of the film-coated working electrodes and a counter electrode, or the potential between each of the film-coated working electrodes and a reference electrode, to obtain a raw data set consisting of a plurality of electrochemical signals; preprocessing the raw data set; and applying chemometric method (s) to the processed data, to qualitatively or quantitively characterize the at least two analytes of interest; wherein the working electrodes are coated with films which contain covalently-bonded ionizable chemical groups, such that the films assume an electrical charge in the test sample. Sensors and processes for preparing the sensors are also disclosed.

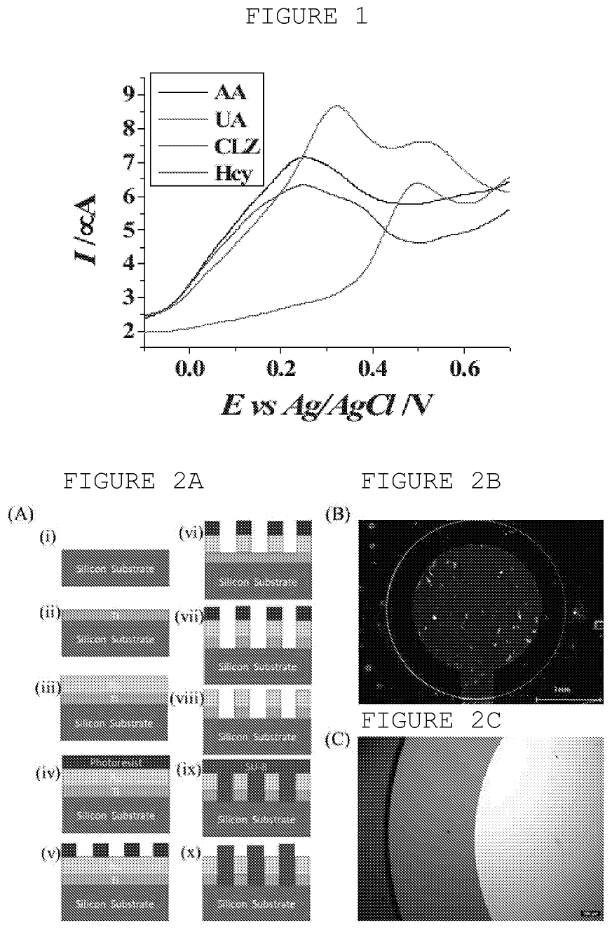

Some analytes in a liquid sample lend themselves to detection by electrochemical techniques, owing to their ability to undergo a redox reaction. A frequently used technique is voltammetry, which is based on a measurement set-up consisting of a working electrode, a counter electrode and optionally a reference electrode, electrically connected to a potentiostat. The current at the working electrode is measured as the potential applied across the working electrode is varied linearly with time. When electroactive species are present in the tested sample, they undergo oxidation (or reduction) when the potential on the working electrode is sufficiently positive (or negative). The oxidation/reduction electrochemical reactions are indicated by an increase in the current (anodic or cathodic) measured; that is, the creation of an electrochemical signal with position and magnitude characteristic of a given analyte. For the purpose of illustration, FIG. 1 is a current versus potential plot (the potential was measured against Ag/AgCl reference electrode) showing electrochemical signatures recorded separately for four molecules at equal concentrations: ascorbic acid (AA; black), uric acid (UA; red), clozapine (CLZ; blue), and L-homocysteine (Hcy; magenta), using a bare gold electrode as the working electrode in a voltammetry measurement cell configuration.

It is often the case that a test sample, for example, a biofluid sample, contains a mixture of redox active analytes. It is not easy to quantify simultaneously the individual components of the mixture, when they possess similar redox potentials, e.g., as shown in FIG. 1. Progress has been achieved with the aid of a novel class of electrochemical sensors, namely, voltammetric electronic tongue sensors. This type of sensor consists of an array of semi-selective electrodes, which allows quantifying multiple analytes simultaneously using data processing algorithms such as principal component analysis, artificial neural networks, and partial least squares regression [D. Ha, Q. Sun, K. Su, H. Wan, H. Li, N. Xu, F. Sun, L. Zhuang, N. Hu and P. Wang, Recent achievements in electronic tongue and bioelectronic tongue as taste sensors, Sens. Actuator. B: Chem. 207 (2015) 1136; X. Cetó, N. H. Voelcker and B. Prieto-Simón, Bioelectronic tongues: New trends and applications in water and food analysis, Biosens. Bioelectron. 79 (2016) 608]. Electronic tongue arrays include several electrodes made of different materials and/or modified with different coatings. For example, the electrochemical sensor disclosed in co-assigned WO 2018/225058 includes a set of coated electrodes that differ from one another in one or more of the following features: film coating material, film coating thickness, film coating density and loading level of conductivity additives incorporated into the film.

We have now found that a mixture of redox species can be analyzed using an array of film-coated working electrodes, to determine the concentrations of individual components of the mixture simultaneously. The differentiation between the redox analytes in the mixture is achieved by manipulating their rates of diffusion from the solution towards the electrodes, across the film coatings. The proposed approach is applicable for mixtures consisting of redox species that differ in size and electrical charge (i.e., ions alongside neutral molecules). With proper selection of film material (e.g., the type of ionizable groups contained in the film, which determines the electrical charge assumed by the film in solution) and control of film thicknesses, the components of such mixtures can be differentiated. That is, the complex electrochemical signal generated by the mixture is separable, owing to differences in electrical charge (sign and magnitude) and molecular weights of the analytes, which ultimately lead to variation in diffusion rates of the analytes. Illustrative mixtures which were successfully resolved into their components contained the redox species tabulated below.

TABLE 1

| | | Ascorbic | | |
|---|---|---|---|---|
| | Fe(CN)₆³⁻ | acid | L-homocysteine | Clozapine |
| Molecular weight | 329.2 | 176.1 | 135.2 | 326.8 |
| (g/mol) | | | | |
| Physiological | −3 | −1 | 0 | +1 |
| charge | | | | |

Accordingly, the invention is primarily directed to an electrochemical method of determining the presence and optionally concentration of at least two analytes in a test sample which contains a mixture of analytes, wherein the analytes differ from one another in electrical charge and/or size, comprising the steps of:
applying variable voltage, fixed voltage, current or impedance across an array comprising working electrodes coated with films of varying thickness and optionally a bare electrode, when the array is in contact with the test sample;
measuring the current flowing or the impedance between each of the film-coated working electrodes and a counter electrode, or the potential between each of the film-coated working electrodes and a reference electrode, to obtain a raw data set consisting of a plurality of electrochemical signals;
preprocessing the raw data set; and
applying chemometric method(s) to the processed data, to qualitatively or quantitively characterize the at least two analytes of interest;
wherein the films contain covalently-bonded ionizable chemical groups, such that the films assume an electrical charge in the test sample.

For example, at least one of the analytes may possess an electrical charge that is opposite to the charge assumed by the films.

The electrodes are preferably made of noble metals, e.g., gold, platinum, rhodium and iridium. Other electrodes, such as glassy carbon electrodes, can also be incorporated into the array of working electrodes. Gold electrodes are generally preferred, for the fabrication of both bare electrodes and film-coated electrodes.

The electrodes are coated with films made of, for example, amino-substituted polymers and carboxylic acid-substituted polymers. Amino-substituted biopolymers (e.g., amino-substituted polysaccharides such as chitosan) assume a positive electrical charge in the test solution because the amino groups undergo protonation. Conversely, acid-substituted biopolymers (e.g., carboxylic acid-substituted polysaccharides such as alginate) become negatively charged in the test solution, because the acidic groups undergo deprotonation.

Films carrying protonatable groups (such as —NH₂) are perhaps more useful in determining the concentrations of analytes which include a negatively charged species. Films carrying de-protonatable groups (—COOH, —SO₃H) may be used in measuring the concentration of analytes which include a positively charged species.

One suitable technique for depositing films onto the electrodes surface is electrodeposition. Chitosan and alginate lend themselves to electrodeposition owing to their pH-dependent hydrogel-forming properties. Films with different thicknesses can be electrodeposited onto electrodes surface by controlling deposition parameters, e.g., electrodeposition time, to produce an array of film-coated electrodes. In the experimental work reported below, the biopolymer chitosan was used to modify the surface of several electrodes, creating films that differ in thickness and density to affect the transport of the analyte across the film towards the electrode. That is, by varying the parameters of the chitosan film, its thickness and pore sizes can be tuned and consequently, can alter the diffusion coefficients of redox molecules through electrostatic interactions and by varying the diffusion paths. This will eventually influence the electrochemical signal generated by the test sample. In turn, the complex electrochemical signal acquired can be treated by the chemometric model's partial least squares regression (PLSR) to differentiate between overlapping electrochemical signals generated from a multicomponent mixture containing several redox-active molecules.

The electrochemical sensor can be fabricated in different sizes and geometries. In WO 2018/225058 an example of an electrochemical sensor with cylindrical geometry was shown, e.g., a cylindrical body made of silicon, polyvinyl alcohol or polydimethylsiloxane, which was 2 to 5 cm long, and with a diameter in the range from 2 to 3 cm. The electrodes were deployed on one base of the tubular body: a reference electrode positioned concentrically and coaxially in respect to the cylindrical body, a ring-shaped counter electrode encircling the reference electrode; and multiple surface modified working electrodes positioned in a radial direction from the reference and counter electrodes and evenly distributed along the perimeter of the base of the cylindrical body. The opposite base provides the electrical wiring to be connected to potentiostat/galvanostat (the electrodes extend along the cylindrical body and are connected to the wiring in the opposite base). When put to use, the electrochemical sensor is simply immersed in a solution to be analyzed such that the base of the cylinder, where the electrodes are disposed, is exposed to the solution allowing the electrodes that (optionally) protrude from the base to be dipped into the liquid sample, creating the electrochemical cell for the measurements.

Now, however, in addition to the design shown in WO 2018/225058, alternatives based on microfabricated configurations are provided for the electrochemical sensor, which are useful because the volume of test samples available for measurements is often very small. Accordingly, a lab-on-chip configuration, i.e., a microsensor device of only millimeters to a few square centimeters (e.g., from 1 mm² to 10 cm²) with electrodes deployed in small chambers for holding the test sample, and a suitable fabrication method, form additional aspects of the invention.

That is, the invention relates to an electrochemical microsensor, for example, in the form of a lab-on-a-chip (and to a method of using it to determine analytes as described herein) comprising:
an array of working microelectrodes, wherein the working microelectrodes include:
one or more bare microelectrodes;
one or more microelectrodes coated with 15 to 35 nm thick film(s) containing covalently-bonded ionizable chemical groups; one or more microelectrodes coated with 35(36) to 50 nm thick film(s) containing covalently-bonded ionizable chemical groups; one or more microelectrodes coated with 50(51) to 65 nm thick film(s) containing covalently-bonded ionizable chemical groups; one or more microelectrodes coated with 65(66) to 80 nm thick film(s) containing covalently-bonded ionizable chemical groups; one or more microelectrodes coated with 80(81) to 100 nm thick film(s) containing covalently-bonded ionizable chemical groups; and optionally one or more reference microelectrodes and a counter microelectrode.

One approach for fabricating the microelectrodes is shown in FIG. 2. It involves a combination of photolithography and etching techniques. The microelectrodes are patterned on a substrate, e.g., a wafer made of oxidized silicon, e.g., a glass substrate or silicon/silicon oxide (A(i)). In the design shown in FIG. 2, each gold-made microelectrode rests on a metallic base attached to the glass or the Si/SiO₂ surface, e.g., a titanium base, which typically corresponds in shape and size to the gold microelectrode, because titanium adheres strongly to the glass substrate. Therefore, deposition of an adhesion layer (A(ii)), e.g., a titanium layer or chromium layer, onto the cleaned substrate (by electron beam evaporation or magnetron sputtering, to create 20 to 30 nm thick adhesion layer) is followed by deposition of the electrode active metal, e.g., 180 to 300 nm thick gold layer, onto the adhesion layer; (A(iii)), also by the same techniques).

Next, individual electrodes are created, protruding from the surface of the substrate and placed within miniature chambers for receiving the liquid sample. For example, disk-shaped electrodes are fabricated, with diameters in the range of 100-1000 microns, or up to 3 mm. To this end, a positive photoresist is applied [(A(iv)), e.g., by spin coating, spray coating or dip coating] on the previously deposited gold layer, followed by soft baking. A first mask is aligned to transfer a pattern, such that on exposure to UV light and development, the exposed areas are removed by being dissolved in, e.g., an alkaline developer (A(v)). Etching solutions can now gain access to defined regions of the previously applied titanium/gold coating (these regions correspond in position and shape to the exposed areas of the positive photoresist). With successive action of suitable etching solutions (first gold etching solution, then titanium etching solution), and removal of the remaining photoresist with the aid of an organic solvent, individual microstructures consisting of the electrode material layer (e.g., gold) atop of the adhesion layer (e.g., titanium) are formed (A(vi)-A(vii)).

The titanium/gold microstructures are surrounded by walls defining an electrochemical chamber for holding a liquid sample. The formation of these chambers is accomplished by photolithography, for example, with the aid of a negative photoresist, applied by spin coating, etc. As shown in FIG. 2, step (A(ix)), the negative photoresist is deposited on, and fills the spaces between, the individual titanium/gold microstructures. After a soft baking step, a suitably designed mask is applied, masking the areas which are atop of the electrode surface, such that on exposure to UV light and development, the unexposed part of the negative photoresist could be dissolved and removed. As a result, the top faces of the electrodes are uncovered, to be accessible to the electrolyte solution. The exposed areas of the negative photoresist remain on the substrate, creating the walls surrounding the titanium/gold electrodes, as shown in FIG. 2, step (A(x)). The developed wafer is washed and dried before it undergoes a hot baking step, followed by cleaning (oxygen plasma). The height of the walls defining the chambers enclosing the electrodes is about 5 to 100 microns. An optical image of the whole microfabricated electrochemical chamber patterned is shown in FIG. 2B. A zoomed-in image of the microfabricated electrode with the negative photoresist-made chamber is shown in FIG. 2C.

FIG. 2 shows just one approach to the structuring of electrodes on a wafer. The application of positive, negative or image reversal photoresists is often a matter of choice and convenience. Also, structuring via etching (i.e., wet etching/dry etching) could be replaced by lift-off procedures. Furthermore, a counter electrode and reference electrodes could be fabricated on the wafer, to be deployed in the vicinity of the working electrodes, or the set of working electrodes may be assembled with commercial counter and reference electrodes (e.g., platinum wire and Ag/AgCl, respectively).

The fabrication of, e.g., Si/SiO₂/Ti/Au device by the method shown in FIG. 2 produces working electrodes exhibiting electrochemical properties and electroanalytical activity comparable to that of commercial gold electrodes. This has been confirmed by cyclovoltammetry in a solution of the ferrocyanide/ferricyanide redox couple [Fe(CN)₆³⁻↔Fe(CN)₆⁴⁻], a commonly used benchmark for assessing newly produced electrodes. Voltammograms recorded for [Fe(CN)₆³⁻↔Fe(CN)₆⁴⁻] redox couple with the fabricated gold layers were fairly similar to those obtained for commercial gold electrodes, showing comparable potentials and peak currents for the oxidation and reduction reactions of the [Fe(CN)₆³⁻↔Fe(CN)₆⁴⁻]. An additional approach for assessing the electroanalytical reliability of the fabricated electrodes involved the calculation of the effective diffusion coefficient of [Fe(CN)₆³⁻] with the aid of the slope of the straight line representing the peak currents versus [Fe(CN)₆³⁻] concentration plots (extracted from voltammograms generated at varying concentrations of [Fe(CN)₆³⁻], as shown in the experimental results reported below). The calculated effective diffusion coefficient of [Fe(CN)₆³⁻] for the fabricated gold electrodes was slower than a literature-reported value (Anal. Chem. 42(1970), p. 1741), presumably due to differences in shape and size between the fabricated electrodes and the gold electrodes reported in the literature. Nevertheless, as pointed out above, despite this difference, similar electrochemical signal characteristics were observed, indicating that the electrochemical activity of the electrodes fabricated by the combination of photolithography and etching, shown in FIG. 2, is acceptable.

Having patterned the electrodes on the substrate, the desired coatings are applied on the gold surfaces, for example, by electrodeposition. In general, the formation of a coating onto the surface of the electrode can be accomplished from a deposition solution by the following electrodeposition techniques:
(i) galvanostatic electrodeposition (chronopotentiometry), in which a constant current is passed across the microelectrode(s) to be coated;
(ii) potentiostatic electrodeposition (chronoamperometry), in which a constant potential is applied on the working microelectrode(s) to be coated; or
(iii) cyclic voltammetry electrodeposition.

The working microelectrodes (divided into groups, as explained above) are modified to create the desired coatings onto their surfaces (each group of working microelectrodes is subjected to surface modification under the same conditions of electrodeposition, to form films of roughly even thickness).

Accordingly, another aspect of the invention is a process for preparing an electrochemical sensor, comprising:
creating microstructures on a substrate (e.g., with the aid of photolithography, etching or both), wherein the microstructure comprises an electrode layer (e.g., a gold layer), optionally disposed atop of an adhesion layer (e.g., titanium layer) attached to the substrate, wherein the microstructures are spaced apart from each other by walls encircling the microstructure and protruding from the surface of the substrate; coating the electrode layers with a film material possessing covalently-bonded ionizable chemical groups, wherein the films created are of varying thickness.

One variant of the process comprises electrodepositing the films onto the electrode layers with different electrodeposition times to influence film thickness. Film thickness depends linearly on electrodeposition times which makes electrodeposition the method of choice in designing the set of electrodes of the invention. For example:
electrodepositing 15 to 35 nm thick chitosan film(s) from a chitosan solution by galvanostatic technique;
electrodepositing 35(36) to 50 nm thick chitosan film(s) from a chitosan solution by galvanostatic technique;
electrodepositing 50(51) to 65 nm thick chitosan film(s) from a chitosan solution by galvanostatic technique;
electrodepositing 65(66) to 80 nm thick chitosan film(s) from a chitosan solution by galvanostatic technique; and electrodepositing 80(81) to 100 nm thick chitosan film(s) from
a chitosan solution by galvanostatic technique.
(film thicknesses reported herein can be measured with the aid of a profilometer or by atomic force microscopy).

Electrodeposited chitosan film-coated microelectrode can be prepared with the aid of a deposition solution with chitosan concentration in the range from 0.5 to 2 wt. %, preferably from 0.8 to 1.2 wt. %, prepared by dissolving chitosan in a strongly acidic environment, whereby the amino groups undergo protonation to reach a slightly acidic pH (5−6). Conductive additives can be included in the deposition solution; these additives will co-deposit and affect the film properties. The concentration of the additives in the deposition solution (e.g., carbon nanotubes (abbreviated herein CNT), gold nanoparticles and platinum nanoparticles) is in the range from 0.1 to 2 wt. %, preferably from 0.8 to 1.8 wt. %. For example, chitosan-CNT electrodeposition solution can be prepared by mixing a chitosan solution as previously described with CNTs, followed by ultra-sonication. The arrayed chip is immersed in the chitosan deposition solution (or chitosan/CNT solution) and electrodeposition is achieved by the chronopotentiometry technique, i.e., selected microelectrodes to be coated are biased to the negative potential against a counter electrode with constant (cathodic) current being applied between the electrodes for a period of time of 15 to 300 seconds, supplied by a DC current source or a galvanostat; typically the current is set in the range from 3 to 6 A/m². A two-electrode configuration is used, i.e., the counter electrode was shorted to the reference terminal. Weakly bound chitosan is removed from the microelectrode surface, by immersing the device in a buffer solution. Below are tabulated electrodeposition times needed to create films with the desired thickness, from ˜1 wt. % by weight chitosan solution with the passage of constant current in the range of 3−6 A/m²:

TABLE 2

| | Electrodeposition time (s) | | | | |
|---|---|---|---|---|---|
| | 15-45 | 45-75 | 75-105 | 105-135 | 135-190 |
| Film Thickness (nm; average) | 15-35 | 35-50 | 50-65 | 65-80 | 80-100 |

As mentioned above, it is possible to incorporate Ag/AgCl reference electrode into the wafer. When on-chip reference Ag/AgCl microelectrode(s) are desired, then it is better to start the surface modification of the multielectrode array with the production of these reference microelectrodes, i.e., by the creation of Ag/AgCl coatings onto one or more microelectrodes. This is achieved via a two-step process; 1) Ag electroplating and 2) Ag anodization in a chloride solution.

In the first step, a suitable Ag electrodeposition solution is prepared by dissolving in water a silver source (e.g., AgNO₃ or Ag₂SO₄, at a concentration from 3 to 10% by weight). Stabilizers such as piperazine which prevent the silver ions from forming nanoparticles can also be added. The solution is made somewhat alkaline (e.g., 8≤pH≤10) by the addition of a weak base such as ammonium hydroxide. The electrodeposition is driven effectively in a continuously stirred Ag⁺ deposition solution (agitation rate is from 50 to 400 RPM), using a two-electrode cell configuration, with the application of a constant current from a DC source or a galvanostat. A cathodic current, fixed in the range from 0.1 to 100 A m², is passed between the electrodes. In case that more than one reference microelectrode is sought to be included in the array, then these microelectrodes are connected simultaneously to the counter electrode to become electroplated with silver (Ag⁰) layer by the reduction of Ag⁺ from the solution. In general, the electroplating lasts a few minutes, usually not more than fifteen minutes, depending on the concentration of the deposition solution, agitation rate, etc.

In the second step, about a quarter to a third of the amount of electrodeposited metallic silver on the microelectrode is converted into AgCl through electrochemical anodization of the silver-coated microelectrodes in a continuously stirred aqueous chloride solution such as hydrochloric acid or sodium chloride, at a constant voltage. A three-electrode configuration can be used, which includes the silver-coated microelectrodes as working electrode(s), Ag/AgCl as reference electrode, and a ring or wire Pt counter electrode. A fixed voltage in the range from 0 to 0.5 V, for example, about 0.2V (vs Ag/AgCl) is applied to the Ag-coated microelectrodes over five to fifteen minutes. Through this two-step process, two or more Ag/AgCl reference microelectrodes are incorporated into the multielectrode array.

Upon completion of the surface modification of the whole set of microelectrodes by the various electrodeposition techniques set out above, the microchip device is rinsed to remove non-deposited material and is ready for use, i.e., to be connected to a potentiostat/galvanostat for electrochemical analysis.

Accordingly, another aspect of the invention is an electrochemical microsensor, comprising:
an array of working microelectrodes in the form of microstructures placed on a substrate, wherein a microstructure comprises an electrode layer, optionally disposed atop of an adhesion layer attached to the substrate, wherein the microstructures are spaced apart from each other by walls encircling each microstructure and protruding from the surface of the substrate; wherein the electrode layers are coated with a film material possessing covalently-bonded ionizable chemical groups, and wherein the array of working microelectrodes is divided into two or more (e.g., three, four or five) groups such that working microelectrodes of different groups are coated with films of different thickness. Each group may include from one to five microelectrodes.

FIG. 9 shows a device incorporating an electrochemical microsensor (1) of the invention. According to the design shown in FIG. 9, the working microelectrodes (3) are placed around a circular-shaped counter electrode (2), equidistantly from the perimeter of the counter electrode. The disk-shaped counter electrode (diameter from 0.5 mm to 2 mm) is surrounded by walls defining a chamber for holding a sample. In the design shown in FIG. 9, a reference electrode (4) is also included.

The device may be powered (5) by a battery or alternatively, can be connected to the main power supply. The microelectrodes of the sensor (1) are connected to a potentiostat and/or galvanostat (6) which control the potential of the working electrodes or the current flowing through the electrodes, respectively, according to the chosen electrochemical technique. In some configurations, the feed of the samples may be fed to the chamber with the aid of integrated pumps or micropump used in microfluidic devices.

The device may further include a data storage unit or a data transmitting unit (7), i.e., a wired transmitter or a wireless network transmitting unit with conventional communication ports to deliver the data to an externally located data storage unit. A data storage unit may be the memory of the data processing unit or any computer readable media. In FIG. 9, personal instruments (9) are shown as well as a cloud-based data storage system (10).

The device further comprises a processor (8) for analyzing a data set of electrochemical signals by one or more chemometric techniques, e.g., multivariate methods such as a supervised machine learning model (artificial neural network (ANN)), or a regression model, e.g., partial least square regression (PLSR).

Briefly, PLSR is a linear regression method and PLSR algorithms are available (e.g., MATLAB). As to ANN, a neural network model is generated with the aid of a training set. To this end, a matrix consisting of a large number of samples with known concentrations of the analyte and with known outputs is collected. As explained in more detail below, the data set is split to create a training set, optionally a cross-validation set and a test set. In the training process, the error between the outputs predicted by the neural network and the known outputs is calculated; this process continues, with the algorithm adjusting the parameters iteratively to minimize the error, i.e., to reduce the error below an acceptable level. Once created, the model is saved and can be used for future measurements of test samples.

It should be noted that raw test data collected by the electrochemical sensor (e.g., a biofluid taken from a patient) undergoes pre-processing with the aid of known techniques before it is fed to the algorithm. Then methods such as principal component analysis (PCA), Fast Fourier Transform (FFT), and selection of important electrochemical signal features, can be used to reduce the dimensions of the data fed to the model. Features selected (e.g., from the voltammograms) include peak current, peak potential, maximum slopes of the I vs. E function (for the increasing and decreasing parts of the function).

That is, to make a measurement of a test sample—using voltammetry for example—the sample is placed in the sample holder in contact with the electrochemical sensor in the device of the invention, as described above, varied voltage is applied by the potentiostat between the reference electrode and working electrode, currents generated are measured and the measurements are stored, and the test data collected (readings from all working electrodes) is preprocessed, reduced and scaled, fed to the algorithm and the concentration of analyte is quantified.

One useful aspect of the invention is that the raw test data collected from a biofluid (i.e., from patients) can be used to calibrate the ANN trained model (that was previously trained using non-biofluid samples, e.g., samples prepared in buffer solutions).

The two approaches for model building—PLSR and ANN are now discussed in more detail; the major steps are outlined below. In both cases, data reduction is based on signal features.

Model Building Process-Based Signal Samples (PLSR)
1. Organization of data in a cell structure—with the aid of MATLAB software reading csv files, all experimental data is arranged in one type of structure (e.g., cell type).
2. Signal smoothing—by using the signal processing toolbox, e.g., MATLAB software, a built-in function (e.g. ‘filter’) was used to filter the signals by employing a moving average window in order to reduce signal fluctuations and noisy behavior which is not originated by the electrochemical properties of the tested solution. A varied filter order in the range of 5<M<8, (M−filter order), depending on the noise level in the recorded data, can be used. In order to keep this parameter unbiased for all the recorded signals in each experiment, it is kept fixed and equal to a specific value for each experimental data.
3. Baseline subtraction—In an electrochemical analysis, the main interest is the faradaic current that is generated owing to the electron transfer from the analyte to the electrode surface in a specific electric potential (oxidation potential). In order to improve the signal to noise ratio (SNR), the Asymmetric least squares spline regression (AsLSSR) can be used. With the aid of MATLAB software, a function is built to estimate the baseline signal by getting two constant values parameters, λ the smoothing parameter (10²<λ<10⁹) and p the asymmetry parameter (0.001<p<0.1). These two parameters take part in the numerical optimization of the cost function of the algorithm.
4. Organization of signals in a matrix structure—the signals are arranged in a matrix form, with each row corresponding to a specific array response. Signals were put in the matrix one after the other, to produce a super row vector structure for each solution, while the target was defined as the concentration matrix, each column describing specific analyte concentration used through the experiments. This has been achieved by building a MATLAB script.
5. Dividing the data set into distinct subsets—The data is separated into two or three distinct sets. The first set is a training set, that is used for the training and the design of the model. All optimization procedures for finding the optimal solution are performed on the training set. It should be noted that the training set could be sub-divided to create a small cross-validation set. The other set is the test set. This set is used to check the model's generalization capabilities, by using the trained model in order to evaluate the ability of the model to predict the concentrations in the “unseen” samples. The data is usually divided as follows: 70−85% of the samples are assigned to the training set (including ˜10% that may be used for cross-validation) and 10−30% for testing. The samples are divided randomly, but the computer's random generation is fixed to assure that the same subdivision could be reproduced.
6. Signals centering—In order to focus on the variability of each specific potential, data is centered, checking the average features value for the all set, and subtracting it from the all signal, resulting in features with mean value equal to 0. The average value of the training set is saved for future use for centering the test set.
7. Choosing a regression model for prediction analysis—the partial least square regression (PLSR) model, a linear technique, is used. It is especially suitable for cases where there is a high correlation between the different features and when there is a limited number of samples (e.g., solutions). The ‘plsregress’ MATLAB function toolbox is used for model building and testing.
8. Choose optimal model parameters (k-fold cross validation)—In order to choose wisely different digital (e.g., number of latent variables in a PLSR model) and physical parameters (e.g., electrode combination), the CV method (LOOCV and 10-fold CV) was used. With the aid of a code that is able to give all the possible configurations without repetition, the CV is implemented in the MATLAB software, using the ‘cvpartition’ function from the statistical toolbox, for random divisions into k sets. By dividing the train set and using it also for validation it is possible to take advantage of most of the information hidden in the data. Model parameters minimizing the cross-validation error were chosen.
9. Model training—The best number of latent variables and best electrode combinations were chosen for training the model on all the training set. A PLSR model using the ‘plsregeress’ function from the MATLAB statistics tool box is built.
10. Test Data pre-processing—The test signals were centered according to the mean average value of the training set.
11. Model predictability—The trained model was used to test and evaluate the performance on unseen data set, i.e., the test set, which was preprocessed and was ready for use as the model input.
12. Evaluate model performance—The quality of the model is assessed with the root mean square error between the known concentrations and those that were estimated by the model.

$${RMSE}_{test} = \sqrt{\frac{1}{{N}_{test}} {\sum }_{i = 1}^{{N}_{test}} {( {C}_{expected} - {C}_{calculated} )}^{2}}$$

(N is the number of samples; C_{expected} is the real actual value and C_{calculated} is the predicted value).

Model Building Process-Based Direct Electrochemical Features (ANN)
1. organization of data in a cell structure—with the aid of MATLAB software, csv files are read, in order to arrange all the experimental data in one type of structure (e.g., cell type).
2. Signal smoothing—by using the signal processing toolbox, MATLAB software, a built-in function (e.g., ‘filter’) is used to filter the signals by employing a moving average window in order to reduce signal fluctuations and noisy behavior which is not originated by the electrochemical properties of the tested solution. A varied filter order in the range of 5<M<8, (M−filter order), depending on the noise level in the recorded data, is used. In order to keep this parameter unbiased for all the recorded signals in each experiment, it was kept fixed and equal to a specific value for each experimental data.
3. Feature extraction—specific electrochemical signal features were extracted, i.e., features which are indicative of the identity of the redox-active molecule and its concentration in the solution. The extracted features include: peak potential, peak current, the maximum slope of the signal, and current value at specific potentials (potentials which are known as the standard oxidation-reduction potential of specific analyte—good evaluation when the peak is not visible). All features are extracted automatically using MATLAB software built-in functions and by customary-built specific functions for each feature.
4. Organize features in a matrix structure—The extracted features were arranged in a matrix form, with each row corresponding to a specific array response, whereas each column describes specific analyte concentration through the experiment. This has been done by building a MATLAB script.
5. Dividing the data set into distinct subsets—The data is separated into two or three distinct sets. The first set is a training set, that is used for the training and the design of the model. All optimization procedures for finding the optimal solution are performed on the training set. It should be noted that the training set could be sub-divided to create a small cross-validation set. The other set is the test set. This set is used to check the model's generalization capabilities, by using the trained model in order to evaluate the ability of the model to predict the concentrations in the “unseen” samples. The data is usually divided as follows: 70−85% of the samples are assigned to the training set (including ˜10% that may be used for cross-validation) and 10−30% for testing. The samples are divided randomly, but the computer's random generation is fixed to assure that the same subdivision could be reproduced.
6. Feature normalization—Features were standardized using the z-score transformation (subtracting the mean value of each feature, and scaling it by dividing the value by the standard deviation). Scaling was preformed because the features were in different scales, such as peak currents [μA] and peak potentials [V]. The data transformation can be achieved with the aid of MATLAB software. The transformation is performed on the training set, when the moments' values were saved for future scaling of the test data.
7. Feature selection—The strategy employed for data reduction to decrease computational complexity is ten-fold cross-validation forward selection based linear regression. The criterion for the selection is the root mean square error between the “real” concentration and those estimated for the validation set. This is achieved with the aid of the statistical toolbox of MATLAB software. In each of the experiments a different initial number of features is used, depending on the technique that is chosen to extract data features.
8. Choosing regression model for prediction analysis—In order to perform multivariate analysis (not only one target value), artificial neural network (ANN) models are used—nonlinear techniques—to explore the relation between the extracted features to the analytes concentration. The ANN MATLAB toolbox is used to explore different network architectures.
9. ANN model optimization (based k-fold cross-validation)—Simple ANN architectures, such as 1-hidden layer with a limited number of neurons, are used in order to reduce the chance for overfitting—the lesser number of neurons in use, the lower network complexity. The best architecture is chosen with the aid of a cross-validation test: the number of neurons in the hidden layer is varied to test the network performance on a validation set. The upper bound of the number of neurons is set such that it is smaller than the number of the model weights. Then the number of neurons with the best score (in terms of the root mean square error between the known concentration and those who were estimated on the validation set) was chosen. The test is repeated with different initial conditions (e.g. different weight initializations) because ANN models are significantly affected by their initial conditions; but in each individual test the parameters are fixed in order to make unbiased and robust decision.
10. Model training—having determined the best architecture, it is now used for training the model across the entire training set. The number of training iterations is limited (early stopping) according to a specific error value that is set to stop the training procedure after reaching at least 99% of the target variance. Hence a trained network that minimizes the performance on the training data is created, ready for future testing.
11. Test data pre-processing—Based on the selected features in the feature selection procedure, the test features were loaded and standardized according to the training moments. For each feature, the training mean value was subtracted, and the result divided by the training standard deviation (this procedure is based on the fact that the two sets sampled from the same data population), creating a scaled data set.
12. ANN predictability—The trained model is used to test and evaluate the performance on unseen data set, i.e., on the test set which was preprocessed and is ready for use as the model input. Calculations are performed in MATLAB software, using the ANN toolbox function and aid function coded for specific tasks.
13. Evaluation of model performance—The quality of the model is assessed with the root mean square error:

$${RMSE}_{test} = \sqrt{\frac{1}{{N}_{test}} {\sum }_{i = 1}^{{N}_{test}} {( {C}_{expected} - {C}_{calculated} )}^{2}}$$

(as previously defined) and the Pearson correlation coefficient (PCC):

$$PCC = \frac{E [ {C}_{expected} - {μ}_{expected} ] E [ {C}_{estimated} - {μ}_{estimated} ]}{{σ}_{expected}^{2} {σ}_{estimated}^{2}}$$

The experimental results reported e ow indicate that mu tip e redox-active molecules with overlapping electrochemical signals can be differentiated with the aid of an array of electrodes fabricated and modified with films that contain protonatable chemical groups (e.g., the positively charged chitosan films) with different thicknesses (e.g., 15−35 nm, 35−50 nm, 50−65 nm, 65−80 nm and 80−100 nm thick film) to differentially affect the diffusion rates of redox-active molecules with different physiological charges. The modified array generated a complex set of electrochemical signals that were resolved into the individual concentrations with the aid of the chemometric PLSR model.

For example, the method of the invention is applicable for a mixture of analytes comprising at least one anion, or a compound dissociating in solution to liberate an anion; and at least one neutral molecule. A mixture of analytes which comprises two or more anions, or compounds dissociating in solution to liberate anions, wherein the anions differ in ionic charge and/or molecular weight, can also be analyzed by the method of the invention. For example, the difference between the molecular weights of the anions is not less than 100 g/mol. Specifically, the mixture of analytes comprises a monovalent anion with a molecular weight below 200 g/mol and a trivalent anion with a molecular weight above 300 g/mol; and a neutral molecule.

In the drawings:

FIG. 1 is a current versus potential plot recorded by voltammetry for four different analytes.

FIG. 2 shows a design of a multielectrode array patterned on a chip (lab-on-chip).

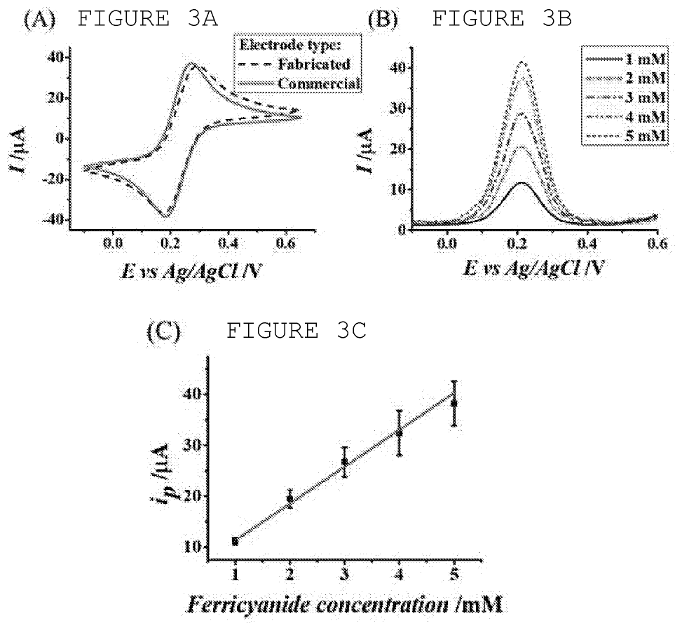

FIGS. 3A-3C show the results collected from voltammetry measurements comparing the gold-fabricated electrode to a commercial electrode in a [Fe(CN)₆³⁻↔Fe(CN)₆⁴⁻] system.

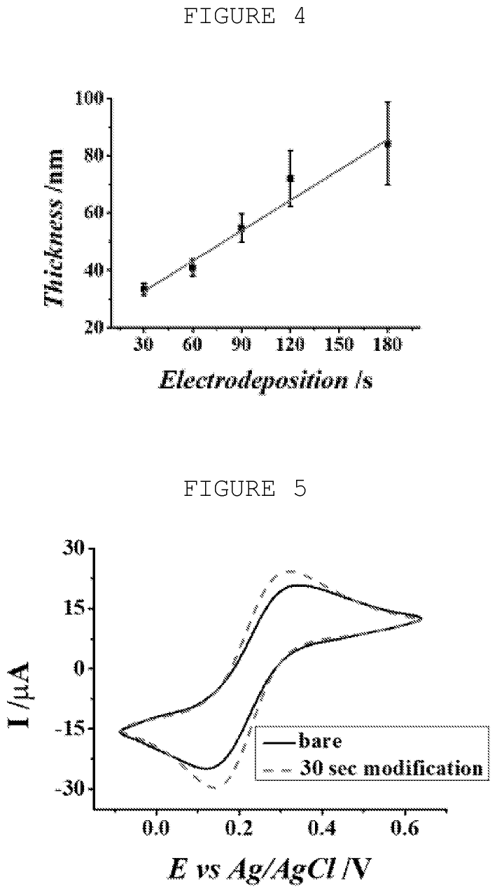

FIG. 4 is the linear relationship between film thickness and electrodeposition time.

FIG. 5 shows the results collected from voltammetry measurements comparing between a surface-modified gold electrode (30 seconds electrodeposited film) and a bare gold electrode in a [Fe(CN)₆³⁻↔Fe(CN)₆⁴⁻] system.

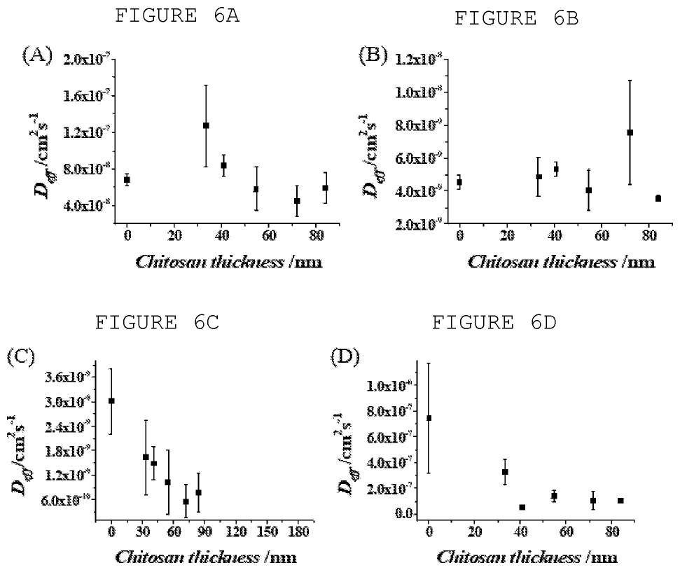

FIGS. 6A-6D show D_{eff} versus film thickness for Fe(CN)₆³⁻, AA, Hcy and CLZ, respectively.

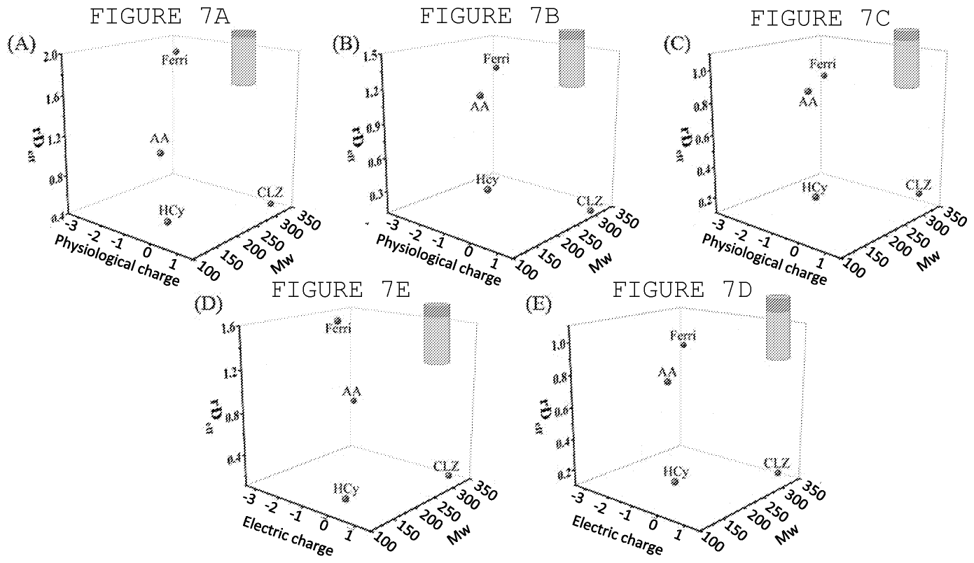

FIGS. 7A-7E show the dependence of rD_{eff} on the physiological charge and the Mw value of the molecules for five different chitosan thicknesses spanning the range from 30 nm to 90 nm.

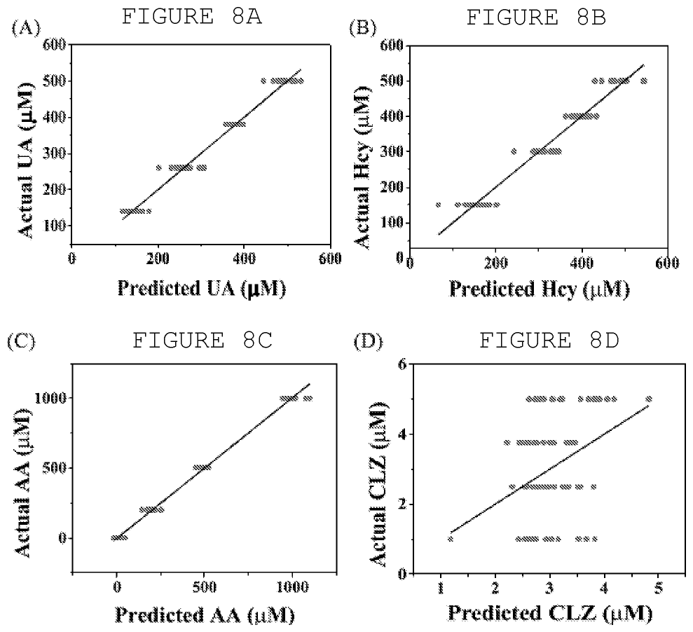

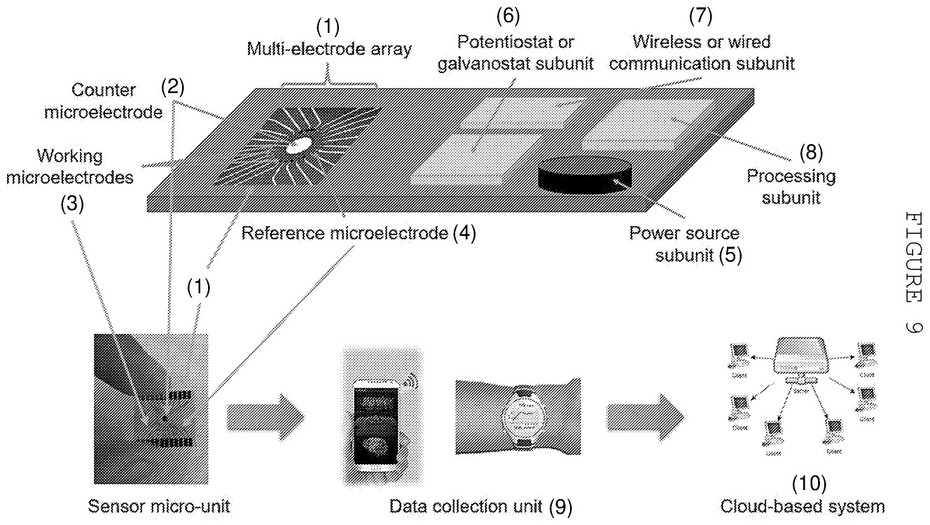

FIGS. 8A-8D show the correlation between the actual concentrations of analytes in a test sample and the concentrations predicted by the method of the invention.

FIG. 9 is a schematic illustration of the device of the invention.

### EXAMPLES

### Example 1Fabrication of an Array of Surface-Modified Gold Electrodes on a Substrate

Five surface-modified chips were produced, each chip with four coated electrodes patterned thereon, by a two-step process: deposition of gold electrodes, and application of coatings on their surfaces.

Step 1: Creating Bare Gold Electrodes

To fabricate an individual chip, four gold disk electrodes (2 mm in diameter) were patterned onto a Si/SiO₂ substrate. A 20 nm-thick layer of Ti was evaporated onto a 4-inch silicon wafer (p-type, orientation: <100>, resistivity: 10−20 ohm·cm, oxide thickness: 500 nm, single side polished, prime grade; University Wafer, Inc.), followed by application of 200 nm-thick layer of gold (E-gun deposition system, VST Service, Ltd.). Next, a positive photoresist (Ti-xlift, MicroChemicals), was spin coated on the wafer (80RC Delta, Universal Spin-coating system, SUSS MicroTec, which was operated at 2200 rpm for 12 s) on the wafer, followed by a soft bake (using a contact hot plate at 110° C. for 2.5 min). The coated wafer was exposed to a designed transparency mask (CAD/Art Services) at a UV light flux of 7.6 mW cm⁻² for 65 s (Karl Suss Mask aligner MA6 system, SUSS MicroTec). The photoresist was then developed in an AZ 726 MIF developer (DEAA174517, Merck) for 8 min. The developed wafer was rinsed in deionized water for 5 min and was dried with nitrogen gas.

An Au wet etching step (10 s long) followed by a Ti wet etching step (6 s long) was used to define the electrodes patterns. The Au etching solution consisted of 4 g of potassium iodide and 1 g iodine dissolved in 40 mL deionized water. The Ti etchant was a commercial Transene solution. The wafer was rinsed in deionized water for 1 min after each etching step. Finally, the remaining photoresist was removed using acetone.

Next, a second photolithography step was conducted to create a chemically stable electrochemical chamber. SU-8 (SU08−3050, MicroChemicals), a negative photoresist, was spanned (80RC Delta, Universal Spin-coating system, SUSS MicroTec, 3000 rpm for 30 s) onto the wafer, followed by a soft bake (using a contact hot plate at 95° C. for 15 min). The coated wafer was exposed to a designed transparency mask (CAD/Art Services) at a UV light flux of 7.6 mW cm⁻² for 50 s (using a Karl Suss Mask aligner MA6 system, SUSS MicroTec), followed by a Post-Exposer Bake (using a contact hot plate at 95° C. for 5 min). The wafer was cooled down to room temperature and then the photoresist was developed in a PGMA ERB developer (DEA165166, Merck) for 8 min and rinsed slowly. The developed wafer was rinsed in deionized water for 5 min and dried with nitrogen gas. After development, the wafer was washed in an IPA solution for 10 s. Then, a hard bake step (using a contact hot plate at 150° C. for 5 min) was followed by an Oxygen Plasma cleaning step (30 W, 500 mtorr, 2 min, 3 sccm). Finally, the wafer was diced into electrochemical testing chips by using a Dicer saw (Dicer ADT-7100, ADT). Prior to the electrochemical testing, cleaning was performed by using acetone, methanol, and isopropanol solution, followed by rinsing with deionized water and drying with a nitrogen gun.

Step 2: Surface Modification of the Electrodes

Chitosan films were electrodeposited onto gold electrodes. The deposition solution consisted of 1% by weight chitosan solution [which was prepared by dilution of a concentrated chitosan solution (1.8%, pH 5.5, obtained by dissolution of chitosan flakes in 2 mol L⁻¹ HCl, to reach a final pH of 5−6, followed by addition of Milli-Q water to a 1% chitosan solution).

A two-electrode set-up was used for a galvanostatic electrodeposition, consisting of a commercial Pt wire counter electrode (CH115, CH Instruments) and the gold working electrode. The chronopotentiometry technique was applied with a current density of 4 A m⁻², I=−15.7 μA (the device was VSP-300 potentiostat, Bio-Logic, Ltd.).

By varying electrodeposition time, films with varying thicknesses were formed on the different chips. Deposition times were 30 s, 60 s, 90 s, 120 s and 180 s for the five surface modified chips. After the modification, each chip was dipped in 10 mM PBS for 1 min to remove any unbound chitosan.

In addition, one chip with four bare electrodes was included in the array, so the total number of working electrodes was twenty-four.

### Example 2Characterization of Gold and Surface-Modified Gold Electrodes

Part A: Gold Electrodes

The purpose of this part of the study was to examine the electrochemical behavior of the fabricated gold electrode (prior to film deposition) and compare it to a commercial gold electrode. The study includes cyclovoltammetry in ferrocyanide/ferricyanide solutions (the [Fe(CN)₆³⁻↔H Fe(CN)₆⁴⁻] redox couple). Cyclovoltammetry tests were performed using VSP-300 potentiostat, Bio-Logic, Ltd., with vertex potential E1=E1=−0.1V vs Ref., vertex potential E2=0.65V vs Ref., scan rate=0.050V s⁻¹ and repeat number of cycles nc=5.

Cyclic voltammograms recorded for the fabricated gold electrode and commercial electrode in 5 mM {Fe(CN)₆³⁻↔Fe(CN)₆⁴⁻} solution indicate a high degree of similarity in voltammograms shape, peak currents and potentials (FIG. 3A; dashed line: fabricated electrode; solid line: commercial electrodes). Peak currents and potentials for the oxidation reaction were 36.1 μA and 0.29V, respectively, for the fabricated electrode and 37 μA and 0.27V, respectively, for the commercial electrode. Peak currents and potentials for the reduction reactions were −36.5 μA and 0.18V, respectively, for the fabricated electrode and −38.2 μA and 0.18V, respectively, for the commercial electrode. Thus, comparable electrochemical characteristics were observed for the fabricated gold electrode and the commercial electrode.

The effective diffusion coefficient of Fe(CN)₆³⁻ for the fabricated gold electrode was also measured. Cyclic voltammograms were recorded (differential pulse voltammetry) using the fabricated gold electrode in {Fe(CN)₆³⁻↔H Fe(CN)₆⁴⁻} solutions with varying concentrations (1 mM, 2 mM, 3 mM, 4 mM and 5 mM of Fe(CN)₆³⁻). Peak currents were acquired; FIG. 3B shows that peak current (iₚ) increases with increasing [Fe(CN)₆³⁻] concentration in the solution. A linear regression analysis of the calibration curve indicates a linear relationship shown in FIG. 3C, in which iₚ versus [Fe(CN)₆³⁻] concentration plot is shown. The linear equation fitted to the straight line was (Eq. 1; R2=0.99):

iₚ=(7.24±0.34)[Fe(CN)₆³⁻]+(4.10±0.52) (1)

The slope of the equation of the straight line was used to calculate the effective diffusion coefficient D_{eff} with the aid of Eq. 2:

iₚ=nCFAD_{eff}(1−expnFΔE/2RT)/πt(1+expnFΔE/2RT) (2)

where iₚ is the peak current value [A], C is the concentration of Fe(CN)₆³⁻[mol cm⁻³], A is the electrode surface area [cm²], D_{eff} is the effective diffusion coefficient of Fe(CN)₆³⁻[cm²·s⁻¹], ΔE is the pulse height of the input signal [V], t is the pulse time [s], n is the number of electrons participating in the electrochemical reaction, R is the universal gas constant [J·K⁻¹·mol⁻¹], F is Faraday constant [A·mol⁻¹], and T is the room temperature [K]. The calculated D_{eff} value of Fe(CN)₆³⁻ for the microfabricated electrode (6.85×10−8±6.49×10⁻⁹ cm² s⁻¹) was two orders of magnitude slower than the one reported in the literature (7.26×10⁻⁶ cm² s⁻¹; see Konopka et al. Anal. Chem. 42 (1970) 1741). The difference in the D_{eff} values may be due to the difference in the shape and size of the electrodes. However, despite this difference, similar electrochemical signal characteristics were observed; therefore, it was concluded that the electrochemical activities of the microfabricated and commercial electrodes are similar.

Part B: Surface-Modified Gold Electrodes

B1) Electrode Surface Characterization:

The chips were dried by using a nitrogen gun. The thickness of the electrodeposited chitosan layer was measured with the aid of a profilometer (Dektak-8, Veeco, Ltd.). The measurements were taken at three different locations on (each of the four electrodes) and the thickness's mean and standard deviation were calculated. Results are tabulated in Table 3 for the five chips (which differ from one another in electrodeposition times and consequently in the thickness of the coatings).

TABLE 3

| | Electrodeposition time (s) | | | | |
|---|---|---|---|---|---|
| | 30 | 60 | 90 | 120 | 180 |
| Film Thickness (nm; average) | 33.4 | 40.9 | 54.7 | 72.0 | 84.1 |

The results are also shown graphically in FIG. 4, in which film thickness is plotted versus electrodeposition time. A linear relationship is observed; the slope of the straight line was calculated to be 0.35±0.04 nm/s.

B2) Cyclovoltammetry Study of Surface-Modified Gold Electrodes:

FIG. 5 shows the cyclic voltammograms recorded for a surface-modified gold electrode (30 seconds electrodeposited film) and the bare gold electrode (CV set-up as described above, i.e., with the {Fe(CN)₆³⁻↔Fe(CN)₆⁴⁻}5 mM solution). Slightly increased oxidation and reductions current peaks were measured by the surface-modified gold electrode (24.27 μA and −29.81 μA, respectively) compared to the bare electrode (20.83 μA and −24.92 μA, respectively), suggesting enhanced sensitivity of a positively charged surface modification (i.e., chitosan film) towards a negatively charged analyte {Fe(CN)₆³⁻↔H Fe(CN)₆⁴⁻}.

B3) Effect of Surface-Deposited Film Thickness on D_{eff:}

The effect of the chitosan thickness on the calculated D_{eff} value for redox molecules of different molecular weights (Mw) and physiological charges was investigated. The results of D_{eff} calculated by the procedures set out above, for five analytes using gold working electrodes (bare, and with varied film thickness), are tabulated in Table 4.

TABLE 4

| | Thickness of chitosan film | | | | | |
|---|---|---|---|---|---|---|
| Redox analyte | bare | 33.4 | 40.9 | 54.7 | 72.0 | 84.1 |
| Fe (CN) ₆³⁻ | 6.9 × 10⁻⁸ ± | 1.3 × 10⁻⁷ ± | 8.4 × 10⁻⁸ ± | 5.8 × 10⁻⁸ ± | 4.5 × 10⁻⁸ ± | 5.9 × 10⁻⁸ ± |
| | 6.5 × 10⁻⁹ | 4.4 × 10⁻⁸ | 1.2 × 10⁻⁸ | 2.4 × 10⁻⁸ | 1.7 × 10⁻⁸ | 1.7 × 10⁻⁸ |
| AA | 4.5 × 10⁻⁹ ± | 4.9 × 10⁻⁹ ± | 5.3 × 10⁻⁹ ± | 4.1 × 10⁻⁹ ± | 7.5 × 10⁻⁹ ± | 3.6 × 10⁻⁹ ± |
| | 3.4 × 10⁻¹³ | 1.2 × 10⁻¹³ | 4.1 × 10⁻¹³ | 1.2 × 10⁻¹² | 3.1 × 10⁻¹² | 1.6 × 10⁻¹³ |
| Hcy | 3.0 × 10⁻⁹ ± | 1.6 × 10⁻⁹ ± | 1.5 × 10⁻⁹ ± | 1.0 × 10⁻⁹ ± | 5.6 × 10⁻¹⁰ ± | 7.8 × 10⁻⁹ ± |
| | 8.0 × 10⁻¹³ | 9.1 × 10⁻¹³ | 4.2 × 10⁻¹³ | 7.8 × 10⁻¹³ | 4.1 × 10⁻¹³ | 4.9 × 10⁻¹³ |
| CLZ | 7.5 × 10⁻⁷ ± | 3.3 × 10⁻⁷ ± | 4.6 × 10⁻⁹ ± | 1.4 × 10⁻⁷ ± | 1.0 × 10⁻⁷ ± | 1.1 × 10⁻⁷ ± |
| | 4.2 × 10⁻¹⁰ | 10 × 10⁻¹¹ | 4.5 × 10⁻¹² | 5.0 × 10⁻¹¹ | 7.1 × 10⁻¹¹ | 2.0 × 10⁻¹¹ |
| UA | 2.1 × 10⁻⁹ ± | 1.8 × 10⁻⁹ ± | 3.3 × 10⁻⁹ ± | 2.0 × 10⁻⁹ ± | 3.3 × 10⁻⁹ ± | 2.3 × 10⁻⁹ ± |
| | 2.2 × 10⁻¹³ | 3.8 × 10⁻¹³ | 2.7 × 10⁻¹³ | 4.6 × 10⁻¹³ | 3.6 × 10⁻¹³ | 3.6 × 10⁻¹³ |

The results are also shown graphically in FIGS. 6A-6D for Fe(CN)₆³⁻, AA, Hcy and CLZ, respectively, in which D_{eff} is shown versus film thickness.

For Fe(CN)₆³⁻ (FIG. 6A), a piecewise relationship was observed. For thin films (in the range of 0 to 30 nm), D_{eff} increases with increasing film thickness. For thicker films (in the range of 30−80 nm), D_{eff} decreases with increasing film thickness. This piecewise relationship suggests that electrostatic attraction between the negatively charged Fe(CN)₆³⁻ and the positively charged chitosan is predominant across the range of small film thickness, whereas thicker chitosan films increase the diffusion path of Fe(CN)₆³⁻, resulting in slower diffusion coefficients.

For the AA, no clear relationship was observed between the D_{eff} and the chitosan thickness (FIG. 6B).

For both Hcy and CLZ, it is seen that D_{eff} decreases with increasing chitosan film thickness (FIGS. 6C & 6D, respectively). Since Hcy is neutral, no attraction forces are expected between the molecule and the chitosan film. Therefore, we can assume that the dominant mechanism affecting the D_{eff} value of Hcy is the denser chitosan film and the increasing diffusion path. In the case of CLZ, which is positively charged, it is assumed that both electrostatic repulsion forces and increased density of the deposited chitosan film account for the negative relationship observed between D_{eff} and film thickness.

B4) Chitosan Films of Varying Thickness can Differentially Affect the Diffusion Rates of Charged Redox Molecules:

Next, the D_{eff} values calculated for the abovementioned redox species (Fe(CN)₆³⁻, AA, Hcy and CLZ) for each chitosan film (i.e., 33.4, 40.9, 54.7, 72.0 and 84.1 nm thick films) were normalized by dividing these values by D_{eff} of the bare electrode, to obtain the relative D_{eff} (rD_{eff}) assigned to molecule X and film thickness Y):

rD_{eff}(molecule X,film Y)=D_{eff}(molecule X,film Y)/D_{eff}(molecule X,bare) (3)

The results are presented graphically in FIG. 7, using three dimensional cartesian coordinate systems to show the dependence of rD_{eff} on the physiological charge and the Mw value of the molecules for different chitosan thicknesses, i.e., for the 33.4 nm-thick chitosan film (FIG. 7A), the 40.9 nm-thick chitosan film (FIG. 7B), the 54.7 nm-thick chitosan film (FIG. 7C), the 72.0 nm-thick chitosan film (FIG. 7D) and the 84.1 nm-thick chitosan film (FIG. 7E).

The results attest to the significance of electrostatic attraction forces between the positively charged chitosan film and the negatively charged redox species Fe(CN)₆³⁻ and AA, with both species showing rD_{eff}>1 (namely, D_{eff} values that are higher than the corresponding values measured for a bare electrode) across a range of film thicknesses, e.g., up to ˜50 nm. A reversal of trend (that is, rD_{eff}>1→rD_{eff}<1) was observed at 54.7 nm thick film for the unipositive AA, and at 84.1 nm thick film for the tripositive Fe(CN)₆⁻³, suggesting that the electrostatic attraction becomes less influential with increasing thickness of the chitosan film, whereby the length of the diffusion path starts playing the major role. Because the physiological charge of Fe(CN)₆⁻³ is higher than for AA (−3 vs. −1), it requires thicker films to decrease the influence of the electrostatic attraction forces in the case of the former compared to the latter.

As to the neutral molecule Hcy and the positively charged CLZ, rD_{eff} values were consistently lower than 1 for all chitosan film thicknesses, in line with the assumptions 1) that the main mechanism affecting the diffusion rate of Hcy is the density of the chitosan film and the length of the diffusion paths, and 2) transport of CLZ is affected by both electrostatic repulsion forces and length of the diffusion path.

### Example 3Simultaneous Prediction of Molecules' Concentration from Mixture Samples

The array of surface modified working electrodes with varying chitosan films thickness of Example 1 was used to acquire a set of diffusion-influenced complex electrochemical signals. The chemometric model PLSR was utilized to analyze the signals.

The experimental set-up consists of twenty four microfabricated working electrodes, a commercial Pt wire as the counter electrode (Platinum counter electrode 23 cm, 012961; ALS Co., Ltd.), and a metal wire coated with Ag/AgCl ink as the reference electrode (Ag/AgCl ink for the reference electrode, BAS, Inc.). Measurements were performed by applying a differential pulse voltammetry (DPV) electrochemical technique (E start −0.1 V vs. Ref, E end 0.7 V vs. Ref., Pulse time 1 ms, Pulse amplitude 55 mV, E step 1 mV, scan rate 10 mV s⁻¹, Equilibration time 10 s; MultiWE32 and Ivium CompactStat potentiostat, Ivium, Ltd.) to all working electrodes simultaneously.

Electrochemical signals were measured by using solutions in which different combinations of the four tested molecules (AA, Hcy, CLZ and UA) were present in 10 mM PBS, i.e., combinations created from the concentrations set out below:
AA: 100, 200, 500, 700, 1000, and 5000 μM.
Hcy: 150, 300, 400, 500, and 600 μM.
CLZ: 1, 2.5, 3.75, 5, and 10 μM.
UA: 140, 260, 380, and 500 μM;

Prior to each measurement, the electrodes were rinsed with 10 mM PBS. The simultaneous measurements with the multi-electrode electrochemical chamber were performed with bare and modified (the abovementioned 30, 60, 90, 120, and 180 s chitosan electrodeposition durations) electrodes. All electrochemical measurements were performed at room temperature.

Chemometric Model PLSR—Training Phase

To train the model, signals for mixtures containing four redox-active molecules at four different concentrations were recorded. A total of 64 solutions was used, with the compositions set out below (the order is CLZ, UA, Hcy and AA).

1(3.75, 260, 500, 500), 2(1, 260, 500, 1000), 3(3.75, 140, 300, 1000), 4(3.75, 380, 400, 500); 5(3.75, 140, 150, 500); 6(2.5, 260, 400, 1000), 7(1, 500, 500, 1000), 8(1, 260, 300, 1000); 9(1, 500, 400, 1000), 10(5, 260, 400, 200), 11(3.75, 140, 300, 200), 12(5, 260, 400, 500); 13(3.75, 140, 400, 200); 14(1, 140, 300, 200); 15(5, 380, 500, 500); 16(1, 260, 300, 200); 17(3.75, 260, 150, 1000); 18(5, 260, 300, 0); 19(3.75, 140, 300, 0); 20(2.5, 140, 400, 500); 21(1, 260, 300, 500); 22(3.75, 380, 300, 1000); 23(5, 140, 400, 200); 24 (3.75, 500, 400, 200); 25(2.5, 380, 300, 500); 26(1, 500, 400, 500); 27(1, 140, 500, 500); 28(3.75, 500, 150, 500); 29(2.5, 260, 150, 500); 30(2.5, 380, 300, 200); 31(1, 380, 150, 500); 32(5, 500, 150, 1000), 33(3.75, 380, 400, 1000); 34(1, 500, 150, 0); 35(3.75, 260, 500, 0); 36(2.5, 260, 400, 0); 37(3.75, 500, 500, 200); 38(3.75, 500, 150, 0); 39(2.5, 140, 150, 1000); 40(1, 140, 500, 0); 41(3.75, 380, 500, 0); 42(2.5, 380, 500, 1000); 43(2.5, 260, 500, 200); 44(1, 140, 400, 0); 45(2.5, 140, 150, 1000); 46(2.5, 380, 500, 0); 47(2.5, 260, 150, 200); 48(1, 380, 150, 200); 49(5, 500, 500, 200); 50(2.5, 500, 500, 200); 51(5, 500, 300, 500); 52 (5, 380, 400, 1000); 53(2.5, 500, 300, 500); 54(1, 380, 400, 0); 55(5, 140, 150, 0); 56(5, 140, 500, 1000); 57(2.5, 500, 400, 0); 58(5, 260, 150, 0), 59(2.5, 500, 300, 0); 60(5, 500, 300, 1000); 61(5, 380, 150, 200); 62(1, 380, 150, 200); 63(5, 140, 500, 500); 64(5, 380, 300, 0).

Testing Phase

The trained model was tested with an additional set of signals measured from 31 mixtures with different concentrations of CLZ, UA, Hcy, and AA. The compositions of the mixtures are set out below (the order is CLZ, UA, Hcy and AA).

1(3.75, 260, 500, 500), 2(5, 500, 150, 1000), 3(3.75, 500, 400, 200), 4(1, 140, 500, 1000); 5(2.5, 380, 300, 500); 6(5, 380, 300, 0), 7(1, 260, 300, 0), 8(1, 380, 150, 200); 9(2.5, 260, 500, 200), 10(2.5, 140, 400, 500), 11(5, 380, 400, 1000), 12(5, 260, 300, 0); 13(3.75, 260, 300, 0); 14(2.5, 380, 300, 200); 15(3.75, 380, 500, 0); 16(5, 260, 150, 1000); 17(3.75, 380, 400, 500); 18 (2.5, 380, 500, 0); 19(1, 380, 400, 0); 20(3.75, 380, 150, 1000); 21(5, 380, 150, 0); 22(5, 260, 150, 500); 23(3.75, 140, 150, 200); 24(5, 260, 400, 500); 25(5, 500, 150, 1000); 26(4.25, 200, 350, 400); 27(4.6, 430, 215, 0); 28(1.5, 300, 460, 700); 29(3.5, 325, 270, 0); 30(2, 400, 420, 900); 31(4.8, 175, 245, 0).

The PLSR model (leave-one-out cross-validation resulted in optimal 12 latent variables; 97.1% cumulative X variance and 98.8% cumulative Y variance) predicted 3 out of the 4 redox-active molecules, as shown by the data tabulated in Table 5.

TABLE 5

| | AA | UA | Hcy | CLZ |
|---|---|---|---|---|
| RMSE | 125 | 51.8 | 55.7 | 1.63 |
| PCC | 0.947 | 0.877 | 0.903 | −0.093 |
| R-squared | 0.894 | 0.753 | 0.809 | 0 |

1. An electrochemical method of determining the presence and optionally the concentration of at least two analytes in a test sample which contains a mixture of analytes, wherein the analytes differ from one another in electrical charge and/or size, comprising the steps of:
applying variable voltage, fixed voltage, current or impedance across an array of working electrodes consisting of electrodes coated with films of varying thickness and optionally a bare electrode, when the array is in contact with the test sample; measuring the current flowing or the impedance between each of the film-coated working electrodes and a counter electrode, or the potential between each of the film-coated working electrodes and a reference electrode, to obtain a raw data set consisting of a plurality of electrochemical signals;
preprocessing the raw data set; and
applying chemometric method(s) to the processed data, to qualitatively or quantitively characterize the at least two analytes of interest;
wherein the working electrodes are coated with films which contain covalently-bonded ionizable chemical groups, such that the films assume an electrical charge in the test sample.

2. The method according to claim 1, wherein at least one of the analytes possesses an electrical charge that is opposite to the charge assumed by the films.

3. The method according to claim 1, wherein the array includes:
one or more working electrodes coated with 15 to 35 nm thick films;
one or more working electrodes coated with 35 to 50 nm thick films;
one or more working electrodes coated with 50 to 65 nm thick films;
one or more working electrodes coated with 65 to 80 nm thick films; and
one or more working electrodes coated with 80 to 100 nm thick films.

4. The method according to claim 1, wherein the films contain protonatable chemical groups.

5. The method according to any on of claim 1, wherein the mixture of analytes comprises:
at least one anion, or a compound dissociating in solution to liberate an anion; and
at least one neutral molecule.

6. The method according to claim 5, wherein the mixture of analytes comprises two or more anions, or compounds dissociating in solution to liberate anions, wherein the anions differ in ionic charge and/or molecular weight.

7. The method according to claim 6, wherein the anions in the analyte mixture differ in ionic charge and molecular weight.

8. The method according to claim 7, wherein the difference between the molecular weights of the anions is not less than 100 g/mol.

9. The method according to claim 8, wherein the mixture of analytes comprises:
monovalent anion with a molecular weight below 200 g/mol; trivalent anion with molecular weight above 300 g/mol; and a neutral molecule.

10. A process for preparing an electrochemical microsensor, comprising:
creating microstructures on a substrate, wherein the microstructure comprises an electrode layer, optionally disposed atop of an adhesion layer attached to the substrate, wherein the microstructures are spaced apart from each other by walls encircling each microstructure and protruding from the surface of the substrate; and
coating the electrode layers with a film material possessing covalently-bonded ionizable chemical groups, wherein the films created are of varying thickness.

11. The process according to claim 10, wherein the microstructures are created by photolithography, etching or both.

12. The process according to claim 10, comprising electrodepositing the films onto the electrode layers with different electrodeposition times to influence film thickness.

13. The process according to claim 12, comprising: electrodepositing 15 to 35 nm thick chitosan film(s) from a chitosan solution by galvanostatic technique;
electrodepositing 35 to 50 nm thick chitosan film(s) from a chitosan solution by galvanostatic technique; electrodepositing 50 to 65 nm thick chitosan film(s) from a chitosan solution by galvanostatic technique; electrodepositing 65 to 80 nm thick chitosan film(s) from a chitosan solution by galvanostatic technique; and electrodepositing 80 to 100 nm thick chitosan film(s) from a chitosan solution by galvanostatic technique.

14. An electrochemical microsensor, comprising:
an array of working microelectrodes in the form of microstructures placed on a substrate, wherein a microstructure comprises an electrode layer, optionally disposed atop of an adhesion layer attached to the substrate, wherein the microstructures are spaced apart from each other by walls encircling each microstructure and protruding from the surface of the substrate; wherein the electrode layers are coated with a film material possessing covalently-bonded ionizable chemical groups, and wherein the array of working microelectrodes is divided into groups such that working microelectrodes of different groups are coated with films of different thickness.

15. The electrochemical sensor according to claim 14, wherein the working microelectrodes are placed around a circular-shaped counter electrode, equidistantly from the perimeter of the counter electrode.

16. The electrochemical microsensor according to claim 14, wherein the array of working microelectrodes comprises:
one or more working microelectrodes coated with 15−35 nm thick film(s) containing covalently-bonded ionizable chemical groups; one or more working microelectrodes coated with 35-50 nm thick film(s) containing covalently-bonded ionizable chemical groups; one or more working microelectrodes coated with 50−65 nm thick film(s) containing covalently-bonded ionizable chemical groups; one or more working microelectrodes coated with 65−80 nm thick film(s) containing covalently-bonded ionizable chemical groups; one or more working microelectrodes coated with 80-100 nm thick film(s) containing covalently-bonded ionizable chemical groups.

17. The electrochemical microsensor according to claim 16, wherein the microelectrodes are coated with chitosan film.

18. A device for electrochemical detection, comprising:
an electrochemical microsensor as defined in any of claim 14; optionally a counter electrode and reference electrode; a potentiostat or galvanostat to which the electrodes are electrically connected to control the potential or current of the working electrodes, respectively, to create a data set of electrochemical signals when the electrodes are immersed in a sample;
a processor configured to analyze the data set of electrochemical signals by one or more chemometric techniques.
